(19) United States
(12) Patent Application Publication
(10) Pub. No.: US 2023/0355509 A1
(43) Pub. Date: Nov. 9, 2023

(54) COMPOSITIONS AND METHODS FOR RELIEVING PAIN

(71) Applicant: Ivar Edward Roth, Newport Beach, CA (US)

(72) Inventor: Ivar Edward Roth, Newport Beach, CA (US)

(21) Appl. No.: 18/355,615

(22) Filed: Jul. 20, 2023

Related U.S. Application Data
(63) Continuation of application No. PCT/US2022/012964, filed on Jan. 19, 2022.
(60) Provisional application No. 63/140,682, filed on Jan. 22, 2021.

(51) Int. Cl. A61K 9/00; A61P 25/02; A61K 45/06

(52) U.S. Cl. CPC A61K 9/0019; A61P 25/02; A61K 45/06

(57) ABSTRACT

The present invention provides a composition and its use in a unique treatment that provides pain relief for mammals. By treating an unconventional set of terminal nerve endings in an extremity (e.g., nerve endings that are generally found in the third plantar web space of the upper and lower extremities), pain in areas proximal to and/or at the root of the nerve can be treated. In several embodiments, these nerve endings are the terminal ends of nerves that emanate from the spinal cord and interconnect with other nerves to supply pain reception to the extremities. Treatment can occur with a variety of modalities to provide temporary and/or permanent pain relief in the unrelated distant part of the body (e.g., those areas closer to the root of the nerve).

### CROSS REFERENCE TO RELATED APPLICATIONS

The present application claims priority to International Patent Application No. PCT/US2022/012964, filed Jan. 19, 2022, published as WO 2022/159480, and which claims the benefit of priority to U.S. Provisional Patent Application No. 63/140,682, filed Jan. 22, 2021. The disclosures of the foregoing applications are hereby incorporated by reference in their entirety. Any and all applications for which a foreign or domestic priority claim is identified in the Application Data Sheet as filed with the present application are hereby incorporated by reference under 37 CFR 1.57.

### Field of the Invention

The present invention provides a composition and its use in a unique treatment that provides pain relief for mammals. In several embodiments, by treating an unconventional set of terminal nerve endings in an extremity (e.g., nerve endings that are generally found in the third plantar web space of the upper and lower extremities), pain in areas proximal to and/or at the root of the nerve can be treated. These nerve endings are the terminal ends of nerves that emanate from the spinal cord and interconnect with other nerves to supply pain reception to the extremities. Treatment can occur with a variety of modalities to provide temporary and/or permanent pain relief in at or near the site of treatment, within a limb comprising the site of treatment, and/or in an unrelated distant part of the body (e.g., those areas closer to the root of the nerve).

### BACKGROUND

### Description of the Related Art

Traditional pain relief treatments have sought to treat the pain points directly at the point of pain (back, foot, hands, groin, etc.). Treatment has included local conservative care modalities, topicals, anesthesia injections, surgical procedures, and/or medicines. These conventional methods have resulted in limited success.

### SUMMARY

Several embodiments pertain to a method of treating pain and/or diagnosing a source of pain. In several embodiments, the method comprises selecting a subject in need of pain management. In several embodiments, the patient is one who has a proximally located source of pain. In several embodiments, the proximally located source of pain comprises one or more of pain in the torso of the body, the shoulder, the upper leg, the head or neck. In several embodiments, the method comprises identifying a web space in the hand or foot of the patient. In several embodiments, the method comprises attenuating nerve function in the web space. In several embodiments, the method comprises attenuating, reducing, and/or limiting pain reception. In several embodiments, nerve function is attenuated using an agent that destroys or disrupts the nerve. In several embodiments, the area where pain is felt is located at a position distant from the area that is treated.

In several embodiments, attenuating the nerve function includes disrupting nerve conduction at the web space. In several embodiments, nerve function is attenuated by subjecting a nerve in the web space to nerve ablation, a chemical treatment, laser therapy, or stem cells. In several embodiments, chemical treatment includes applying a local anesthesia, a steroid, diluted alcohol, or combinations thereof to the nerve in the web space. In several embodiments, nerve function is attenuated by disrupting nerve conduction through injecting a chemical composition into the web space. In several embodiments, the chemical composition comprises, consists of, or consists essentially of ethanol, local anesthesia, a steroid, water, or combinations of any of the foregoing.

Several embodiments pertain to a method of treating pain in the lower body (mid to lower back, pelvis, buttocks, etc.) or lower body extremities (e.g., the hip, knee, and ankle joints, and the thigh, leg, and foot, etc.). In several embodiments, the pain is in the lower body. In several embodiments, the method includes selecting a subject suffering from pain. In several embodiments, the method comprises selecting a subject suffering from pain in the lower body or lower body extremities. In several embodiments, the method comprises attenuating nerve function in a foot of the subject thereby treating the pain. In several embodiments, nerve function is attenuated by subjecting a nerve in the foot to nerve ablation, a chemical treatment, laser therapy, regenerative medicine, or combinations of the foregoing. In several embodiments, the pain is Sciatica pain, lower back pain, hip pain, buttock pain, groin pain, iliotibial band pain, or knee pain.

In several embodiments, the site where nerve function is attenuated includes the anastomotic branch between the common digital nerve to the third web space and common digital nerve to the fourth web space. In several embodiments, the nerve function is attenuated by injecting a chemical composition into the web space. In several embodiments, the chemical composition comprises ethanol, local anesthesia, a steroid, water, or combinations of any of the foregoing.

In several embodiments, the pain is Sciatica. In several embodiments, the pain is lower back pain. In several embodiments, the pain is associated with a bulging, ruptured, or herniated disc or a degenerative disc disorder. In several embodiments, the disc is located in the T10-L5-S1 area of the spine.

Several embodiments pertain to a method of treating pain in the upper body (mid to upper back, shoulders, etc.), the head or neck, or upper body extremities (e.g., the upper arm, forearm, hand, etc.). In several embodiments, the method includes selecting a subject suffering from pain. In several embodiments, the method includes selecting a subject suffering from pain in the upper body, the head and/or neck, or upper body extremities. In several embodiments, nerve function in a hand of the subject is attenuated thereby treating the pain. In several embodiments, nerve function is attenuated by subjecting a nerve in the hand to nerve ablation, a chemical treatment, laser therapy, regenerative medicine, or combinations of the foregoing.

In several embodiments, the pain is in the upper body. In several embodiments, the pain is neck pain, shoulder pain, upper back pain, or head pain. In several embodiments, the pain is head pain caused by temporal mandibular joint pain, migraine, or headache. In several embodiments, the pain is associated with a bulging, ruptured, or herniated disc or a degenerative disc disorder. In several embodiments, the disc is located in the C1 to T9 area of the spine.

In several embodiments, nerve function is attenuated by injecting a chemical composition into a web space in the hand. In several embodiments, the chemical composition comprises, consists of, or consists essentially of ethanol, local anesthesia, a steroid, water, or combinations of any of the foregoing.

In several embodiments, the site where nerve function is attenuated includes the anastomotic branch between the common digital nerve to the third web space and common digital nerve to the fourth web space.

In several embodiments, the subject is a human. In several embodiments, the subject is a companion animal (e.g., a domesticated or domestic-bred animals). In several embodiments, the subject is a dog or a cat. In several embodiments, the subject is a horse.

Several embodiments pertain to a composition for treating and/or diagnosing a source of pain located proximal to the site of treatment, the composition comprising local anesthesia, a steroid, diluted alcohol, or combinations thereof.

### BRIEF DESCRIPTION OF THE DRAWINGS

FIGS. 1A and 1B provide anatomical drawings of a portion of the nervous system of a subject.

FIG. 2A provides an anatomical drawing the spine and spinal nerves.

FIG. 2B provides the regions innervated by the spinal nerves.

FIG. 3 provides an anatomical drawing of nerves of the foot.

FIG. 4 provides an anatomical drawing of nerves of the hand.

### DETAILED DESCRIPTION

Several embodiments disclosed herein pertain to compositions and methods of using those compositions for the treatment of pain (and/or stiffness, lack of range of motion, or other issues associated with pain). As disclosed herein, it has been surprisingly found that treating a site (e.g., manipulating nerves at a site) that is distal from a site of pain alleviates or lessens pain despite the site of treatment being a distance away from (and/or at area unrelated to) the site of pain. In several embodiments, the site of pain is proximal to the body of the subject while the site of treatment (e.g., where one or more nerves is disrupted and/or where nerve function is attenuated) is located distally (e.g., further from torso and/or the center of the body than the site of pain). In several embodiments, despite nerve activity being attenuated at the site of treatment, fine motor skills and/or coordination is not affected. In several embodiments, nerve attenuation comprises turning off pain receptors at the site of treatment (e.g., at as site of injection) without disrupting other normal functions of the nerve at the site of treatment. For instance, though pain receptors have been turned off, the site of treatment may still have normal sensory perception (e.g., it is not numb or the patient does not perceive numbness) and/or motor skills and coordination are not affected (or are improved). In several embodiments, treatment is performed at a time when the patient is suffering pain (e.g., stiffness, lack of range of motion, etc.). A variety of compositions and methods of treating pain are described below to illustrate various examples that may be employed to achieve one or more desired improvements. These examples are only illustrative and not intended in any way to restrict the general inventions presented and the various aspects and features of these inventions. Furthermore, the phraseology and terminology used herein is for the purpose of description and should not be regarded as limiting. No features, structure, or step disclosed herein is essential or indispensable. Any feature, structure, component, material, step, or method that is described and/or illustrated in any embodiment in this specification can be used with or instead of any feature, structure, component, material, step, or method that is described and/or illustrated in any other embodiment in this specification. Additionally, any feature, structure, component, material, step, or method that is described and/or illustrated in one embodiment may be absent from another embodiment.

Terms and phrases used in this application, and variations thereof, especially in the appended claims, unless otherwise expressly stated, should be construed as open ended as opposed to limiting. As examples of the foregoing, the term “including” should be read to mean “including, without limitation, ” “including but not limited to, ” or the like; the term “comprising” as used herein is synonymous with “including, ” “containing, ” or “characterized by, ” and is inclusive or open-ended and does not exclude additional, unrecited elements or method steps; the term “having” should be interpreted as “having at least; ” the term “includes” should be interpreted as “includes but is not limited to; ” the term “example” is used to provide exemplary instances of the item in discussion, not an exhaustive or limiting list thereof; and use of terms like “preferably, ” “preferred, ” “desired, ” or “desirable, ” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of”will be understood to include those elements specifically recited and those additional elements that do not materially affect the basic and novel characteristics of the claimed technology. The phrase “consisting of”excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

As used herein, “and/or” refers to and encompasses any and all possible combinations of one or more of the associated listed items, as well as the lack of combinations when interpreted in the alternative (“or”).

As used herein, the term “sciatica” refers to pain that radiates along the path of the sciatic nerve, which branches from your lower back through your hips and buttocks and down each leg. Typically, sciatica affects only one side of your body though it may affect both (e.g., the left and right) sides of the body.

The term “effective amount, ” as used herein, refers to that amount of a recited compound and/or composition that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, an effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some embodiments, an improvement in a condition can be a reduction in disease symptoms or manifestations (e.g., pain, anxiety & stress, seizures, malaise, inflammation, mood disorders, insomnia, etc.). Actual dosage levels of active ingredients in an active composition of the presently disclosed subject matter can be varied so as to administer an amount of the active compound(s) that is effective to achieve the desired response for a particular subject and/or application. The selected dosage level will depend upon a variety of factors including, but not limited to, the activity of the composition, composition, route of administration, combination with other drugs or treatments, severity of the condition being treated, age, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

“Treat” or “treating” or “treatment” refers to any type of action that imparts a modulating effect, which, for example, can be a beneficial effect, to a subject afflicted with a disorder, disease or illness, including preventing the manifestation of disease states associated with the condition, improvement in the condition of the subject (e.g., in one or more symptoms or in the disease), delay or reduction in the progression of the condition, and/or change in clinical parameters, disease or illness, curing the illness, etc.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient. ” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects. Where site of treatment is disclosed with respect to a human, the equivalent site of treatment for an animal can be used.

As used herein, the term “weight percent, ” when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

As used herein, the term “volume percent” (or “percent by volume, etc.) when referring to a component, is the volume of the component divided by the volume of the composition that includes the component, multiplied by 100%. For example, the volume percent of component A when 5 milliliters (mL) of component A is added to 95 mL of component B is 5% (e.g., 5 mL A/(5 mL A+95 mL B)×100%).

As used herein, when the term “collectively or individually” (and variations thereof) modifies an amount of a component or components (e.g., a weight percent) of multiple component composition, this usage means that each individual component may be provided in the amount disclosed or that combined amount of components may be provided in the amount disclosed. For example, if agents A and B are referred to as, collectively or individually, being present in a composition at a wt % of 5%, that means that A may be at 5 wt % in the composition (individually), B may be at 5 wt % in the composition (individually), or the combination of A and B may be present at a total of 5 wt % (A+B=5 wt %, e.g., collectively). Where A is present at 5 wt %, B may be absent. Where B is present at 5 wt %, A may be absent. Alternatively, where both A and B are present, A may be at 5 wt % (individually) and B may be at 5 wt % (individually), totaling 10 wt % (collectively).

### Introduction

Traditional pain relief treatments have sought to treat the pain points directly at the point of pain (back, foot, hands, groin etc.) with various types of treatments including, local conservative care modalities, topicals, anesthesia injections, surgical procedures, and/or medicines. Conventional treatments have also included treating pain occurring with nerve blocks performed at the root of a nerve and/or at positions more proximal to the spinal cord than the location within the body that is experiencing pain. For example, injecting medicine to block nerve activity at or near the spinal cord to alleviate sciatic pain, pain in the leg, etc. This is thought to be useful for pain that radiates from the lower back into a leg, such as that caused by disc herniation or spinal stenosis (narrowing around the nerves) which triggers nerve root irritation. Similar injections are thought to be useful in the cervical spine, where the symptoms (including pain) may extend into the arms. In many instances, these treatments have resulted in limited success.

Disclosed herein are methods of addressing pain in novel and unexpected ways. In several embodiments, disclosed herein are methods that use remote pain relief treatments. Remote pain relief treatments, as disclosed herein, include where an area is treated that is not at the site of the pain and would appear to not be an area to treat. In several embodiments, in reference to the location of the spinal cord, the area of treatment in the remote pain relief treatment is more distal than the site at which pain is occurring. In several embodiments, during treatment a unique combination of terminal nerve endings is targeted for treatment. This method has not previously been considered as a pain relief solution until now. The methods disclose herein yield unexpected and surprising pain relief results.

### Methods of Treating

Several embodiments pertain to methods of treating pain. In several embodiments, the pain is a pain occurring at or near the spine and/or spinal cord (e.g., in the torso, neck, upper legs, shoulders, upper arms, etc.). The spinal cord is the bundle of nerves that carries messages back and forth from the brain to the muscles and other soft tissues. As the spinal cord travels down the back, it is protected by a stack of backbones called vertebrae (which also hold the body upright). From the spinal cord, nerves extend from a position proximal to the spinal cord distally out to the extremities, including, for example, muscles in the arms, hands, legs, and feet. These nerves extend from the spinal cord through openings between the vertebrae. In several embodiments, the pain can have a number of origins, including but not limited to muscular skeletal acute injury, post-operative pain (e.g., after a surgical procedure), arthritis, structural damage through use (e.g., sports injuries, pulls, sprains, strains), etc.

In several embodiments, as disclosed elsewhere herein, methods of treating pain are provided. In several embodiments, the method includes attenuating nerve function (e.g., disrupting, lowering the amount of, and/or eliminating nerve function) at a site of administration. In several embodiments, the site of nerve attenuation (e.g., disruption of nerve function) is at an area distal to the site of the pain (and/or discomfort) that is to be treated. For example, in several embodiments, using the spinal cord as a reference point, the area and/or site of nerve disruption may be located within the foot (e.g., distally located) to treat a site of pain or discomfort in the buttock or upper leg (e.g., proximally located pain). To illustrate, to treat pain located in the buttock (more proximally located), a nerve within the foot (more distally located) is disrupted and/or treated. To treat pain located in the neck (more proximally located), a nerve within the hand (more distally located) is disrupted and/or treated. In several embodiments, a nerve in an area of an extremity (e.g., the hand or foot) is manipulated to achieve treatment of pain at a location more proximally located with respect to the body of the subject. In several embodiments, a proximally located area of pain or discomfort is one that is closer to the spine or trunk of the body than the distally located site of treatment. In some embodiments, as disclosed elsewhere herein, manipulating the nerve function at a site distal to the area of pain or discomfort allows treatment of the proximally located area of pain or discomfort (e.g., a lessening or elimination of the pain or discomfort, improved mobility and/or function, less stiffness, etc.).

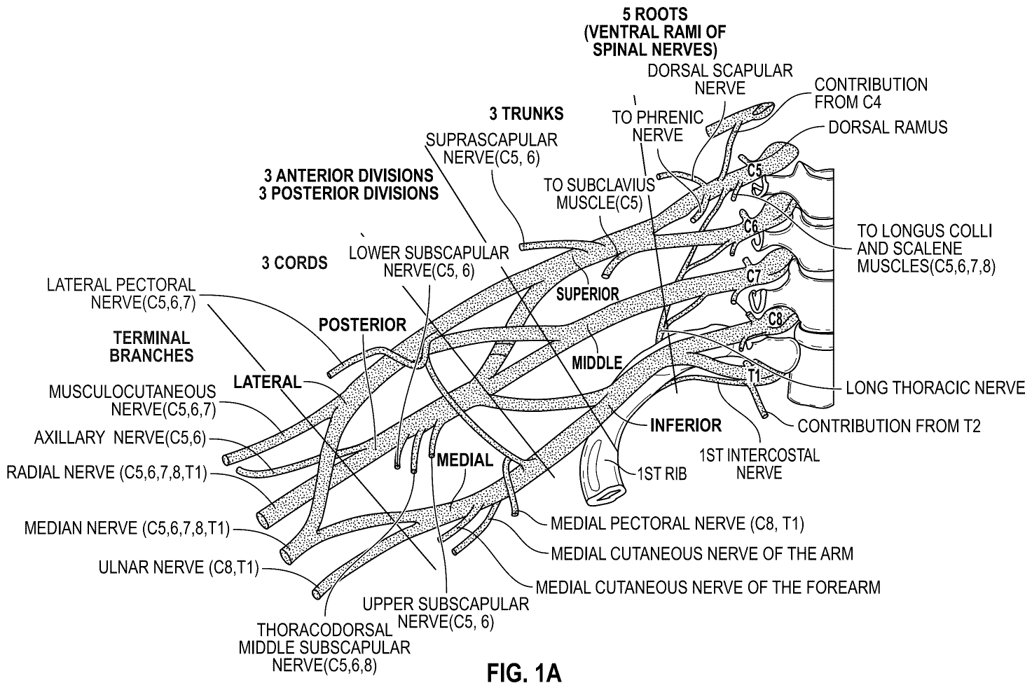

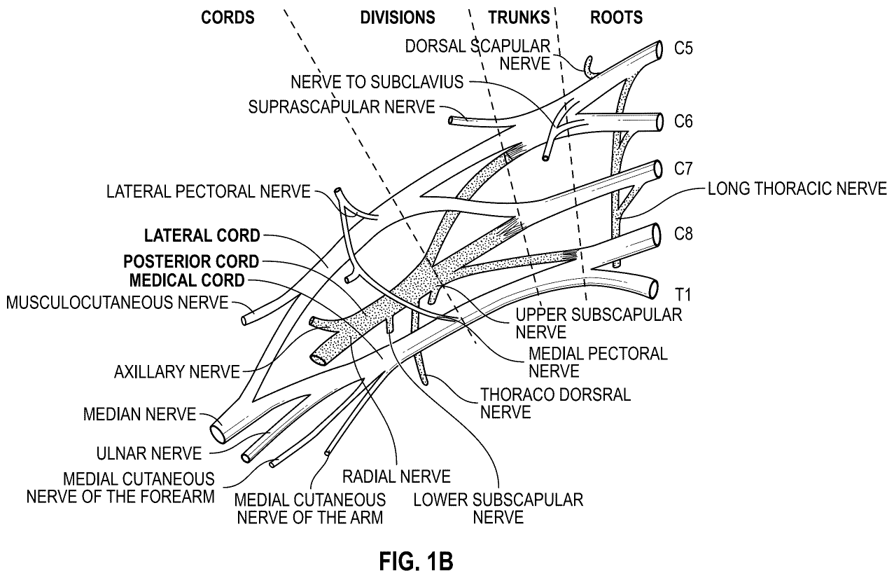

FIGS. 1A and 1B provide anatomical diagrams of nerves extending from the spinal cord, providing the root, which then forms nerve trunks, followed by divisions, then cords, then terminal branches, and then nerve endings. In several embodiments, the nerve is attenuated at a position at or more proximal to the nerve endings than the spinal cord. For example, if the site of pain is found proximal and/or at the trunk of a nerve, then the site of treatment may be at a cord or nerve ending, etc.

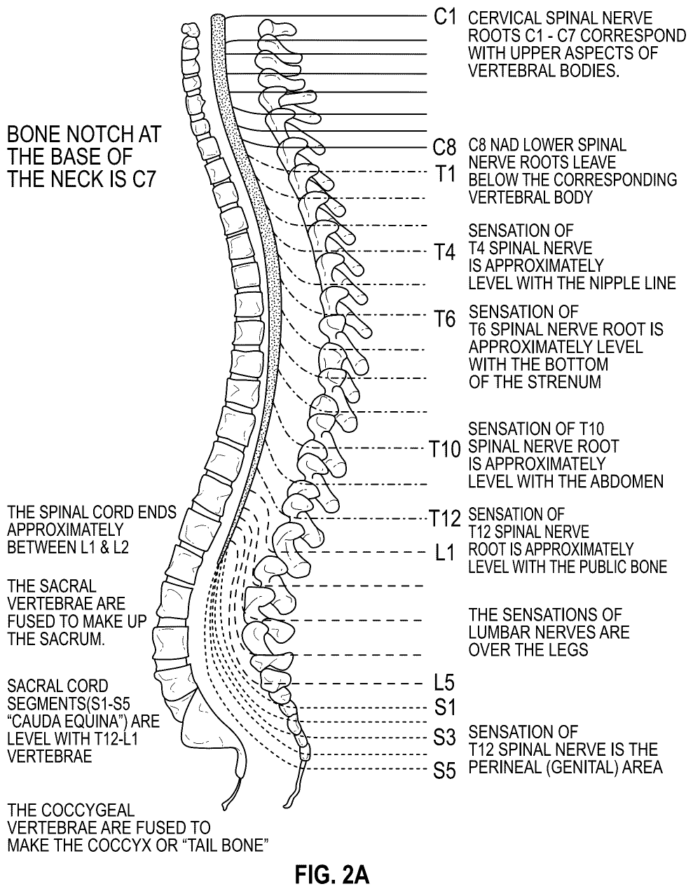

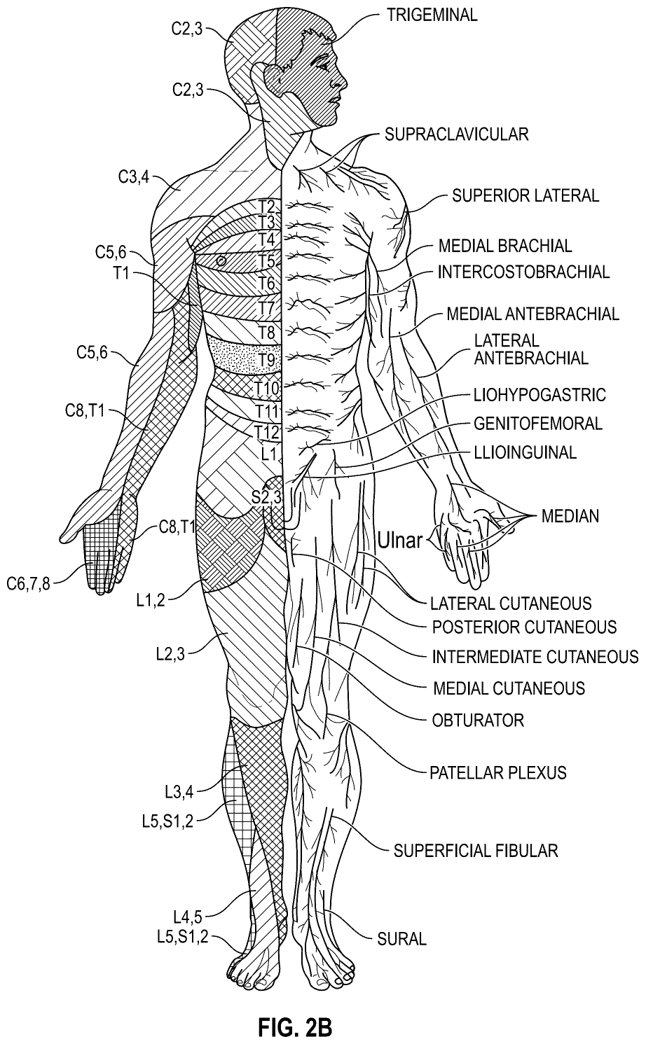

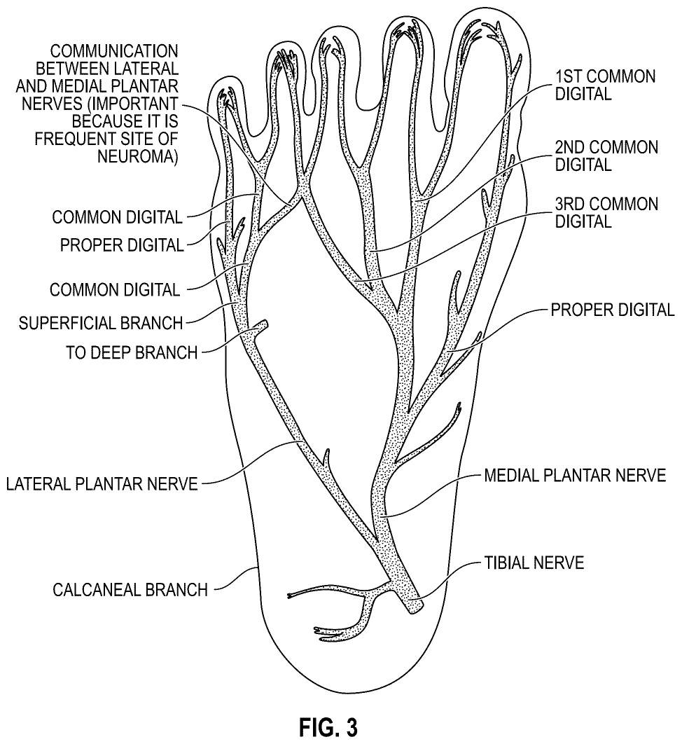

A spinal nerve is a mixed nerve, which carries motor, sensory, and autonomic signals between the spinal cord and the body. As shown in FIG. 2A, in the human body there are 31 pairs of spinal nerves, one on each side of the vertebral column. These are grouped into the corresponding cervical (C1 to C8), thoracic (T1 to T12), lumbar (L1 to L5), sacral (S1 to S5) and coccygeal regions of the spine. In several embodiments, the pain to be treated is a pain associated with nerves from C1-T5 (e.g., shoulder, neck, upper back, etc.). In several embodiments, when the pain to be treated is associated with C1-T5 originating nerves, the site of treatment is an upper extremity (e.g., the arm or hand). In several embodiments, the pain to be treated is a pain associated with nerves from T10-L5-S1 (e.g., buttock, hip, upper leg, mid back, lower back, etc.). In several embodiments, when the pain to be treated is associated with T10-L5 originating nerves, the site of treatment is a lower extremity (e.g., the leg or foot).

In several embodiments, the nerve to be treated (e.g., attenuated) for lower extremity pain is in the foot. In several embodiments, as disclosed elsewhere herein, the site of treatment may be a lower extremity. In several embodiments, where pain is associated with nerves originating from T10-L5-S1 of the vertebral column, the site of treatment (e.g., where a nerve is attenuated and/or manipulated) is located between, adjacent to, or proximal to the metatarsal bones (and/or to a metatarsal bone) in the foot. In several embodiments, the site of treatment (e.g., where a nerve is attenuated and/or manipulated) is between the first and second metatarsal. In several embodiments, the site of treatment (e.g., where a nerve is attenuated and/or manipulated) is between the second and third metatarsal. In several embodiments, the site of treatment (e.g., where a nerve is attenuated and/or manipulated) is between the third and fourth metatarsal. In several embodiments, the site of treatment (e.g., where a nerve is attenuated and/or manipulated) is between the fourth and fifth metatarsal. In several embodiments, the location in the foot to be treated (e.g., where a nerve is attenuated and/or manipulated) is the anastomotic branch between the common digital nerve to the third web space and common digital nerve to the fourth web space. It has now been noted that this is a communication point between the lateral and medial plantar nerves in the foot which eventually course, intertwine and incorporate with the nerves representing the lumbar and sacral plexus. It has now been noted that this is the typical area where injury and arthritis to the spine occurs for the back and from which the sciatic nerve emanates from. It has now been found that practically all if not all pain in the extremity emanates from these plexuses of nerves. Single common digital nerves may also be treated in the other web spaces or where a terminal branch is located. In several embodiments, single common digital nerves may also be treated in the other web spaces or where a terminal branch is located but due to the limited nerve innervation, the extent of the relief is limited to a small or single nerve root in the affected spine. In several embodiments, where pain is associated with nerves originating from T10-L5-S1 of the vertebral column, the site of treatment (e.g., where a nerve is attenuated and/or manipulated) is located adjacent to the first metatarsal, the second metatarsal, the third metatarsal, the fourth metatarsal, the fifth metatarsal, or combinations of any of the foregoing.

In several embodiments, if the pain is emanating from one side of the body the treatment (e.g., where a nerve is attenuated and/or manipulated) is administered to that corresponding side of the body. For example, as in one sided sciatica, the extremity on the side of the body affected by sciatic pain is treated (e.g., a nerve in an extremity on that side of the body is attenuated and/or manipulated). Alternatively, if the pain is distributed substantially equally about, for example, the medial plane of the body, both extremities may be treated to alleviate the pain. For example, in several embodiments, for back pain centered in the lower spine, treatments may be given to both extremities (e.g., given to both feet resulting in the relief to the entire lower back). In other embodiments, if the pain is distributed substantially equally about, for example, the medial plane of the body, a single extremity may be treated (e.g., by nerve attenuation) to alleviate the pain.

In several embodiments, in rare occasions limited or no response can be seen with treatment to the 3rd interspace and this is most probably due to an anatomical variation which occurs in a small percentage of the population or the injection not being properly administered.

In several embodiments, as disclosed elsewhere herein, the site of pain may be an upper extremity (and/or a portion of the body associated with nerves originating from C1-T5 of the vertebral column). In several embodiments, where pain is associated with nerves originating from C1-T5 of the vertebral column, the site of treatment for such pain is the hand. In several embodiments, where pain is associated with nerves originating from C1-T5 of the vertebral column, the site of treatment (e.g., nerve attenuation) is located between or proximal to metacarpal bones (and/or to a metacarpal bone) in the hand. In several embodiments, the site of treatment is between the first and second metacarpal. In several embodiments, the site of treatment is between the second and third metacarpal. In several embodiments, the site of treatment is between the third and fourth metacarpal. In several embodiments, the site of treatment is between the fourth and fifth metacarpal.

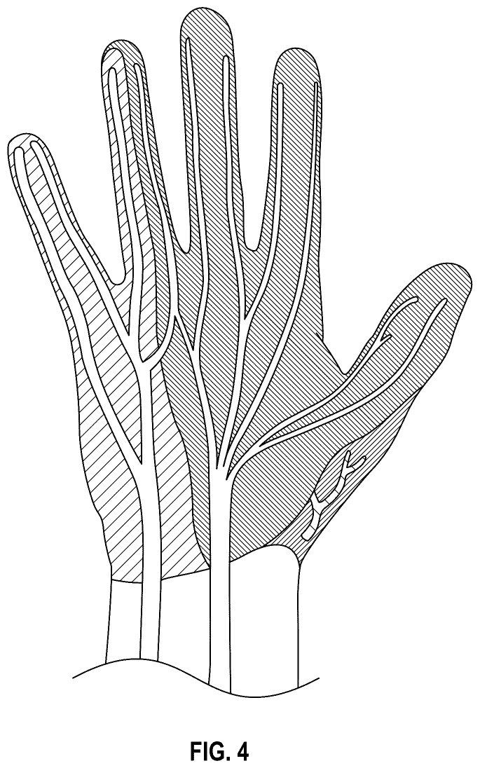

In several embodiments, as disclosed elsewhere herein, the nerve anatomy treated for upper extremity pain is in the hand and is analogical to the foot where the communicating branch is found in the third interspace of each hand. In several embodiments, this is where the common digital branch of the third and fourth digital nerve communicate from the median and ulnar nerves (as shown in FIG. 4).

In several embodiments, the area covered from by hand nerve attenuation (e.g., injection) correlates to the brachial plexus in the same way as the lower extremity with additional nerves communicating with the median and ulnar nerves as they course upwards and become the brachial plexus. In several embodiments, this affords a potential wide area of pain relief in the upper extremity. In several embodiments, similar to the foot, if there is a one-sided pain the corresponding hand is treated. If the neck itself is the problem both hands may be treated simultaneously.

In several embodiments, examples of lower extremity complaints indicated for this treatment include Sciatica pain, lower back pain, hip pain, buttock pain, groin pain, iliotibial band pain knee pain, ankle, and foot pain (or combinations of the foregoing). In several embodiments, examples of lower extremity complaints indicated for this treatment include tightness of the lower back and/or numbness and tingling of the leg and foot. In several embodiments, examples of upper extremity complaints indicated for treatment are neck pain, migraines, shoulder pain, elbow pain, hand, and thumb pain. In several embodiments, examples of upper extremity complaints indicated for this treatment include stiff neck, loss of range of motion to the shoulder/arm, and weakness of the entire upper extremity including the hand, wrist, and thumb, etc. In several embodiments, the pain treated is back pain (e.g., lower back, mid-back, or upper back) or neck pain. In several embodiments, the methods disclosed herein treat pain and stiffness in the neck, back, or lower back. In several embodiments, the methods disclosed herein treat burning pain that spreads to the arms, buttocks, or down into the legs (sciatica). In several embodiments, the methods disclosed herein treat numbness, cramping, or weakness in the arms, hands, or legs. In several embodiments, the methods disclosed herein treat loss of sensation in the feet. In several embodiments, the methods disclosed herein treat difficulty with hand coordination.

In several embodiments, the pain treated using one or more methods disclosed herein is a pain due to compression of the spinal cord. Spinal cord compression can occur anywhere from your neck down to your lower spine and cause symptoms, such as numbness, pain, and weakness. Depending on the cause of the compression, symptoms may develop suddenly or gradually, and they may require anything from supportive care to emergency surgery. As disclosed elsewhere herein, the spine is divided into three primary areas (and the sacral iliac area). The cervical spine is the upper part of your spine that supports your neck. The cervical spine has seven vertebrae. The middle part of your back is your thoracic spine. It is made up of 12 vertebrae. The lumbar spine is your lower back and has five vertebrae. The sacral iliac area consists of the sacrum and iliac bond, within the pelvis. Spinal compression can occur in any of these areas. One of the most common causes of spinal compression is the gradual wear and tear of osteoarthritis. People who develop spinal cord compression from this are usually older than 50. In several embodiments, the techniques disclosed herein can be used to alleviate pain caused by osteoarthritis. Other conditions that may cause spinal cord compression can develop more quickly, even very suddenly, and can occur at any age. In several embodiments, the spinal compression can originate from any of the following conditions: osteoarthritis, abnormal spine alignment, injury to the spine, bone disease, rheumatoid arthritis, or combinations of any of the foregoing.

In some embodiments, pain is quantified by measuring pain during (e.g., turning or nodding the head for neck pain, bending at the hip for back pain, etc.) using one or more of the following pain scales: Visual Analog Scale for Pain (VAS Pain), Numeric Rating Scale for Pain (NRS Pain), McGill Pain Questionnaire (MPQ), Short-Form McGill Pain Questionnaire (SF-MPQ), Chronic Pain Grade Scale (CPGS), Short Form-36 Bodily Pain Scale (SF-36 BPS), and Measure of Intermittent and Constant Osteoarthritis Pain (ICOAP). In some embodiments, in patients receiving treatment, pain decreases by equal to or at least about: 5%, 10%, 25%, 50%, 75%, 90%, or ranges including and/or spanning the aforementioned values.

In several embodiments, the method comprises diagnosing a site of pain. In several embodiments, a site distal to the site of pain is then located. In several embodiments, the site to be treated that is distal to the site of pain is a site having nerve endings that are downstream from nerves at the site of pain.

In several embodiments, a test treatment is performed prior to nerve attenuation. In several embodiments, the test treatment can be performed using the therapeutic compound, local anesthesia, or combinations thereof. For example, a small amount of nerve attenuating agent (e.g., a partial dose) or a partial regimen may be performed to determine if a patient is a candidate for treatment. In several embodiments, for example, where the therapeutic is injectable, an initial injection of a therapeutic compound is given to test the site of treatment. In several embodiments, the therapeutic compound is a local anesthesia (or another agent that attenuates nerve function as disclosed herein). In several embodiments, if the results are positive with the test treatment (e.g., pain is lowered at the site of pain) than a therapeutic treatment can be performed. In several embodiments, the therapeutic treatment includes a series of treatments (e.g., a series of alcohol sclerosing injections).

In several embodiments, a series of alcohol sclerosing injections (or other nerve attenuating treatments) is performed and used for long term benefits (e.g., efficacy for equal to or greater than 3 months, 6 months, 9 months, one year). In several embodiments, a series of alcohol sclerosing injections (or other nerve attenuating treatments) is performed to achieve permanent benefits. In several embodiments, the injections (or other nerve attenuating treatments) are usually given and/or performed weekly until the desired results are obtained. In several embodiments, one or more extra administrations (e.g., injections) are performed after the injection that has caused the full extent of the desired relief. In several embodiments, relief is usually found using 4 to 7 weekly injections (although in a more or less weekly injections may be used to achieve results). In several embodiments, the administration is performed weekly (e.g., once a week). In several embodiments, the administration is performed multiple (e.g., 2, 3, 4, 5, or more) times in a single week. In several embodiments, treatment may be performed by administrations (e.g., multiple administrations) over a period of time equal to or less than: 1 week, 2 weeks, 3 weeks, 4 weeks, 5 weeks, 6 weeks, 7 weeks, 2 months, 3 months, or ranges spanning and/or including the aforementioned values.

In several embodiments, symptomatic relief using the test injections ranges from ten seconds to about ten minutes. Sometimes results continue to improve and take a few hours but typically this process is almost instantaneous.

In several embodiments, the patient provides a history of their pain (complaint). In several embodiments, once it has been determined where the problem exists (e.g., the site of pain and/or the symptom to be treated) than either one or both sides are injected. In several embodiments, the area is cleaned with disinfectant (e.g., alcohol) prior to treatment. In several embodiments, a local anesthetic (e.g., a numbing agent, such as a cold refrigerant topical spray) is applied to the injection site prior to the injection being given.

For acute injury situations, like in an emergency room or first presenting initially after the injury it may be advisable to use a local anesthetic plus a steroid mixed together. In several embodiments, this combination will provide immediate relief from the anesthetic with a longer acting duration from the steroid providing from days to many weeks of relief. In several embodiments, administering a combination will decrease or eliminate the need or use of opiates and or anti-inflammatory medicines from being prescribed with their concomitant side effects and or abuse potential. In several embodiments, combination treatments may be used for chronic and/or persistent pain as well.

For lower extremity problems it has been found that biomechanically controlling the foot causes the foot mechanics to improve by controlling pronation. This in turn makes the more proximal joints knee, hip and vertebra work more effective and efficient and in its own right helps with controlling the abnormal motions in the lower extremity which causes the alignment to improve thus help taking the abnormal stresses off of the lower back and helping structurally to control and lessen the effects of poor foot posture which will help with problems like lower back, knee and Sciatica pain. In several embodiments, an orthotic is used. In several embodiments, the orthotic is a full contact, dynamic orthotic which flexes, and acts as a shock absorber when the foot is in contact with the orthotic thus controlling abnormal motions and allowing the foot to self-correct and improve its function in a dynamic way.

Other applications include using this treatment for diagnostic techniques before elective surgeries. An example of this is if a patient had hip pain, a treatment (e.g., nerve attenuation) would be performed to determine if in fact the surgery would be indicated. If the pain went away from the treatment, surgery could be avoided or delayed.

In several embodiments, the methods disclosed herein are useful in treating Parkinson's Disease, fibromyalgia, Sudeks atrophy/reflex sympathetic dystrophy, or a symptom of any of the foregoing. In several embodiments, for example, tremors associated with Parkinson's Disease are lessened. In several embodiments, pain associated with fibromyalgia and/or Sudeks atrophy/reflex sympathetic dystrophy is lessened as disclosed elsewhere herein.

In several embodiments, the pain relief associated (and/or other symptom relief) with a treatment lasts for a period of greater than or equal to about: 1 week, 2 weeks, 1 month, 2 months, 6 months, 1 year, 2 years, or ranges including and/or spanning the aforementioned values. In several embodiments, the pain relief is substantially permanent. In several embodiments, the pain is reduced by 50% (using a pain scale as disclosed herein (e.g., a reduction from 8 on a pain scale to 4 on the same pain scale, etc.)) for a period of greater than or equal to about: 1 week, 2 weeks, 1 month, 2 months, 6 months, 1 year, 2 years, or ranges including and/or spanning the aforementioned values.

### Compositions and Techniques for Treatment

Examples of treatments to attenuate nerves (e.g., for the third interspace terminal nerve endings) can vary and can include any technique used to disrupt nerve function. In several embodiments, the technique used to attenuate nerve function includes one or more of nerve ablation, local anesthesia, steroids, diluted alcohol, laser therapy, stem cells and/or any treatment which can affect nerve conduction or inflammation of the affected nerve. Also included is any treatment for repairing the affected nerves with regenerative medical means and surgical repair or release of the affected nerves by open or closed surgical methods. The nerve attenuating agent may be a local anesthetic, alcohol, phenol, steroid, or combination thereof. In some embodiments, the nerve is frozen or heated to ablate it.

In several embodiments, as disclosed elsewhere herein, the composition (e.g., the injectable nerve attenuating composition) comprises alcohol. In several embodiments, the alcohol is ethanol. In several embodiments, the ethanol used is dehydrated alcohol (e.g., 200 proof). In several embodiments, the alcohol is diluted with a local anesthesia. In several embodiments, the ethanol is paired with a local anesthesia (or multiple local anesthetics) in varying concentrations (e.g., from 4 to 10% by volume alcohol). However lower or higher concentrations may be used. In several embodiments, the % by volume of ethanol in the injectable composition is equal to or less than about: 1%, 2%, 4%, 6%, 8%, 10%, 15%, 20%, 25%, 30%, 35%, 40%, or ranges including and/or spanning the aforementioned values.

As disclosed elsewhere herein, the alcohol may be diluted with a local anesthesia. Any local anesthesia can be used by itself or as a mixture of local anesthetics. The local anesthesia (or mixture of local anesthetics) may be used with or without epinephrine. In several embodiments, the local anesthesia comprises an active agent selected from the group consisting of lidocaine, mepivacaine, prilocaine, bupivacaine, etidocaine, ropivacaine, levobupivacaine, marcaine, Carbocaine, Akten, articaine, ropivacaine, septocaine, nupercainal, Naropin, tetracaine, chloroprocaine, dibucaine, isocaine, or combinations of any of the foregoing. In several embodiments, the active agent(s) (collectively or individually) is present in the local anesthesia at a weight percent of equal to or less than about: 0.1%, 0.5%, 1%, 2%, 3%, 5%, 7.5%, or ranges including and/or spanning the aforementioned values. In several embodiments, the local anesthesia is aqueous (e.g., includes water, saline, etc.) and/or comprises water. In several embodiments, the weight percent of water in the local anesthesia equal to or at least about: 99.9%, 99.5%, 99%, 98%, 97%, 95%, 93%, 90%, 85%, 80%, or ranges including and/or spanning the aforementioned values. In several embodiments, the % by volume of local anesthesia (including combinations of multiple anesthetics) in the composition (e.g., the injectable nerve attenuating agent) is equal to or at least about: 75%, 80%, 85%, 88%, 90%, 92%, 94%, 96%, 98%, or ranges including and/or spanning the aforementioned values. The local anesthesia can be any injectable local anesthesia with or without epinephrine of varying strengths usually 1 to 2%. In several embodiments, the % by volume of epinephrine in the injectable composition is equal to or less than about: 0.5%, 1%, 2%, 4%, 6%, or ranges including and/or spanning the aforementioned values.

The targeting of a unique combination of terminal nerve endings in the hands and feet of human beings or the appropriate appendage in other mammals to provide relief of pain in other parts of the body is unique and inventive. In several embodiments, the injection of specialty medicines to the terminal nerve endings to provide temporary or permanent pain relief. In several embodiments, type of medicines used for attenuating nerve function include but are not limited to local anesthetics, steroids, stem cells, regenerative medicines, and diluted alcohol injections.

In several embodiments, a dehydrated alcohol solution is injected (e.g., the volume of ethanol in the injectable composition is equal to or less than about 1%, 2%, 4%, 6%, 8%, 10%, 15%, 20%, 25%, 30%, 35%, 40%, or ranges including and/or spanning the aforementioned values). In several embodiments, the injection is performed in the webspace using an appropriately sized needle approximately 1 to 1.5 inches in length using ½ to 1 cc of solution depending on the size of the foot or hand and the age of the individual. In several embodiments, a dorsal approach can be used approximately one inch in from the webspace. In several embodiments, higher concentrations can be used. In several embodiments, the injection is performed using up to 10% or greater than 10% dehydrated alcohol (e.g., the volume of ethanol in the injectable composition is equal to or less than about 1%, 2%, 4%, 6%, 8%, 10%, 15%, 20%, 25%, 30%, 35%, 40%, or ranges including and/or spanning the aforementioned values). In several embodiments, this will cause a painful reaction due to a superficial infiltration of the agent dorsally rather than central to the plantar aspect where the nerve is located. If irritation occurs an oral anti-inflammatory and or ice can be used to treat the site of treatment. The burning or pain usually resolves in a day or two.

The attenuating agent composition can be formulated for subcutaneous injection. The pharmaceutical composition can be in the form of a liquid, a suspension, a dispersion, or a solution. The compound(s) can be in admixture with a suitable carrier, diluent, or excipient such as sterile water, physiological saline, or the like, and can contain auxiliary substances such as wetting or emulsifying agents, pH buffering agents, or viscosity enhancing additives, preservatives, and the like, depending upon the preparation desired. See, e.g., “Remington: The Science and Practice of Pharmacy”, Lippincott Williams & Wilkins; 20th edition (Jun. 1, 2003) and “Remington's Pharmaceutical Sciences, ” Mack Pub. Co.; 18^{th} and 19^{th} editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts.

Suitable inert materials include diluents, such as carbohydrates, or inorganic salts such as calcium triphosphate, calcium phosphate, sodium phosphate, calcium carbonate, sodium carbonate, magnesium carbonate, and sodium chloride.

### Kits

The compositions of some embodiments can be provided to an administering physician or other health care professional in the form of a kit. The kit is a package which may house a container which contains an active compound in a suitable pharmaceutical composition, and instructions for administering the pharmaceutical composition to a subject. The kit can optionally also contain one or more additional active compositions (e.g., for nerve attenuation). The kit can also contain separate doses of a compounds and/or compositions of some embodiments for serial or sequential administration. The kit can optionally contain one or more diagnostic tools and instructions for use. The kit may comprise a device to allow ablation of a nerve to be attenuated. The kit can contain suitable delivery devices, e.g., syringes, and the like, along with instructions for administering the compound(s). The kit can optionally contain instructions for storage, reconstitution (if applicable), and administration of any or all compounds or compositions included. The kit can include a plurality of containers reflecting the number of administrations to be given to a subject. In several embodiments, a kit for the treatment of pain is provided that includes the compositions described herein or another formulation and one or more therapeutic agent currently employed for the treatment of pain.

### EXAMPLES

### Example 1

Upper extremity examples Case #1. A Female 53 years old was seen complaining of shoulder pain to both shoulders which caused her difficulty sleeping at night due to pain over the last several months. She also had a stiff neck and mild neck pain. With her left shoulder she had limited range of motion of her arm when raising it upward. even though she was going to physical therapy. On her right side she had pain in the arm and extreme weakness to the point she was unable hold a toothbrush to brush her teeth and needed two hands to hold a cup when attempting to use her right hand. Ten minutes after the injection she had less pain to all areas and within 24 hours she was pain free and able to brush her teeth and pick up a glass with the right hand.

### Example 2

Case #2 A female aged 78 complained of unrelenting pain of 40 years duration to her neck, this occurred after an auto accident in her 30′s. She had sought many types of intervention but to no avail. This included medical doctors, neurologist's orthopedics, and pain doctors. Her MRI and Cat scan showed C2-C7 disk extrusions and narrowing of the spinal canal. She was prescribed anti-inflammatory medicines, codeine, many sessions over the years of physical therapy, traction, Chiropractic, etc. She also suffered from severe migraines. She was told that she would just have to live with her neck problem. Both hands were injected and within a few minutes here neck pain was completely gone. After her series of injections, she has remained pain free in her neck and now does not have any more Migraines. She stated that she did not know what it was like to live pain free with her mood dictated by the degree of pain she was experiencing.

This patient also suffered from lower back pain and sciatica. Her treatments included epidural's, stem cells, traction, physical therapy, chiropractic, yoga, and exercise to no avail. She had bilateral injections into her feet with the test solution of local anesthetic after which within ten minutes her symptoms were totally relieved. She had the series of injections on a weekly basis and after seven injections has not had any pain for the last four months. Along with the injections she wears her dynamic orthotics every day in all her shoes.

### Example 3

Case #3. This 62-year-old male complained of long standing chronic arthritic pain and stiffness to his right thumb. He had previously seen an orthopedic surgeon, had a PRP injection to his thumb, steroid pain shots, 60 days of physical therapy rehab, and several laser treatments etc. All treatments were not satisfactory up to this point. After his injection to the third interspace of his right hand he had immediate full relief of his pain and stiffness to his thumb. This is an example of Brachial plexus Involvement as the nerves involved in the interspace injected do not innervate the thumb, however they do connect to the radial nerve at the level of C8 supplying the thumb area and therefore stop the pain signal.

### Example 4

Lower extremity example Case #1. This 70 y/o male had a long-term history of lower back pain with Sciatica and had eight previous back surgeries. At this time, he still had debilitating pain which was controlled with 6 Percocet's daily and only able to walk and sit for short periods of time. A test shot was given to him which resolved 70% of his symptoms immediately. Over the course of his bilateral injections his Sciatica symptoms have resolved entirely. His lower back pain has resolved 80%. Currently the patient has gone from walking a quarter of a block to over two miles and he has lost 30 pounds over the last 5 months. He recently took a walk for 8.5 miles. He can now drive his car for many hours something he could not do before. He only takes Percocet when he goes to sleep to avoid some morning stiffness.

He always wears his orthotics.

### Example 5

Case #2. This 73 y/o male had a long history of back pain and as of two years ago had back surgery. Currently his back pain is returning limiting his ability to enjoy his life. His operating doctor has advised him that there is nothing more he can do except send him to the pain doctor. The patient had the test shot and experienced 100% relief immediately. After this he had his series of seven injections in both feet and he has been pain-free ever since.

### Example 6

Case #3 This female aged 73 had debilitating hip pain for many years and was treating with a Chiropractor and massage. She stated that the adjustments would usually last a day or so before she was in pain again. Her pain was so severe that she recently fell breaking her wrist. A test injection was given into her left foot and the pain immediately went away. She was able to walk with no pain and had no stumbling when she would get up from a sitting position. This patient underwent her weekly injections for 7 weeks and is now pain free and able to exercise for the last 5 months.

### Example 7

This example demonstrates results of treatment of an acute injury. The patient was a 64-year-old male nurse who recently complained of sciatica from lifting a heavy patient. The subject was given a local with steroid and this resolved all his complaints when he returned two weeks later. He therefore was not a candidate for the series of injections as his acute symptoms had resolved.

### Example 8

This example demonstrates the diagnostic aspect of the injection. This 81-year-old male had a left hip replacement 3 years ago but still suffers from the same type of pain as before his hip replacement he also suffered from Sciatica. He had an injection to his left foot and had immediate relief of pain for his left hip and his sciatica pain was gone. He continued with the series of injections and orthotics and has had complete pain relief for the last 6 months. This is a case where probably unnecessary surgery was performed for his left hip pain which most likely was back related referred pain.

### Example 9

This 48 year old Female had suffered for decades with fibromyalgia affecting her neck and lower back. She took Imuran and anti-inflammatories for her pain and discomfort. Test Injections were given to her hands and feet and within ten minutes she was pain free with all stiffness gone. The test injections gave her relief for about a week.

### Example 10

This 65-Year-old male suffers from diabetes and severe Parkinson's disease-causing significant tremors of his hands and cramping of his forearms from the muscle spasms of the tremors. He is on medicine to control his Parkinson's, yet the tremors sometimes are very severe. A test injection was given to both hands and within a few minutes the patient stated that his arms felt lighter and the tremors were reduced by 60%.

### Example 11

This is a prophetic example. A 60-Year-old male suffers from Sundek's atrophy/reflex sympathetic dystrophy. In this disease the nerves are in a hyperexcited state and so the pain perceived to the patient is out of proportion to the injury. A test injection is given to the hands and/or feet of the effected extremity and within a few minutes the patients pain would be reduced or eliminated.

1. A method of treating pain and/or diagnosing a source of pain, the method comprising:
selecting a subject in need of pain management and having a proximally located source of pain;
identifying a web space in a hand or foot of the subject; and
attenuating nerve function in the web space in the hand or foot of the subject;
wherein a site of treatment is located at a position distal to the source of pain.

2. The method of claim 1, wherein attenuating the nerve function includes disrupting nerve conduction at the web space.

3. The method of claim 2, wherein nerve function is attenuated by subjecting a nerve in the web space to nerve ablation, a chemical treatment, laser therapy, or regenerative medicine (e.g., with stem cells).

4. The method of claim 3, wherein the chemical treatment includes applying a local anesthesia, a steroid, diluted alcohol, or combinations thereof to the nerve in the web space.

5. The method of claim 2, wherein attenuating the nerve function includes disrupting nerve conduction by injecting a chemical composition into the web space.

6. The method of claim 5, wherein the chemical composition comprises ethanol, local anesthesia, a steroid, water, or combinations of any of the foregoing.

7. A method of treating pain, the method comprising:
selecting a subject suffering from pain; and
attenuating nerve function in a foot of the subject thereby treating the pain;
wherein nerve function is attenuated by subjecting a nerve in the foot to nerve ablation, a chemical treatment, laser therapy, regenerative medicine, or combinations of the foregoing;
wherein the pain is Sciatica pain, lower back pain, hip pain, buttock pain, groin pain, iliotibial band pain, or knee pain.

8. The method of claim 7, wherein the site where nerve function is attenuated includes the anastomotic branch between the common digital nerve to the third web space and common digital nerve to the fourth web space.

9. The method of claim 8, wherein nerve function is attenuated by injecting a chemical composition into the web space.

10. The method of claim 9, wherein the chemical composition comprises ethanol, local anesthesia, a steroid, water, or combinations of any of the foregoing.

11. The method of claim 7, wherein the pain is Sciatica.

12. The method of claim 7, wherein the pain is lower back pain.

13. A method of treating pain, the method comprising:
selecting a subject suffering from pain; and
attenuating nerve function in a hand of the subject thereby treating the pain;
wherein nerve function is attenuated by subjecting a nerve in the hand to nerve ablation, a chemical treatment, laser therapy, regenerative medicine, or combinations of the foregoing;
wherein the pain is neck pain, shoulder pain, upper back pain, or head pain.

14. The method of claim 13, wherein the pain is head pain caused by temporal mandibular joint pain, migraine, or headache.

15. The method of claim 14, wherein nerve function is attenuated by injecting a chemical composition into a web space in the hand.

16. The method of claim 15, wherein the chemical composition comprises ethanol, local anesthesia, a steroid, water, or combinations of any of the foregoing.

17. The method of claim 13, wherein the site where nerve function is attenuated includes the anastomotic branch between the common digital nerve to the third web space and common digital nerve to the fourth web space.

18. The method of claim 1, wherein the subject is a human.

19. A composition for use in treating and/or diagnosing a source of pain located more proximal to a body of a patient than a site of treatment with the composition, the composition comprising local anesthesia, a steroid, diluted alcohol, or combinations thereof.

20. The method of claim 13, wherein the subject is a human.
